(19) United States
(12) Patent Application Publication
(10) Pub. No.: US 2024/0105319 A1
(43) Pub. Date: Mar. 28, 2024

(54) CARE PROVIDER ROUTING SYSTEMS

(71) Applicant: AndorHealth, LLC, Orlando, FL (US)

(72) Inventors: Maha Ahmed, Orlando, FL (US); William Beamer, Orlando, FL (US); Sonali Carumbaya, Pleasanton, CA (US); Lauren Cuppy, Windermere, FL (US); Declan Fortune, Richmond, TX (US); Jack Gilmore, Windermere, FL (US); Siddarth Kasi, Windermere, FL (US); Neha Mahmood, Orlando, FL (US); Nishanth Reddy, Windermere, FL (US); Chanakya Toleti, Windermere, FL (US); Chandini Toleti, Windermere, FL (US); Kalyan Toleti, Windermere, FL (US); Tatiana Vallejo, Saint Cloud, FL (US); Andrew Langford, Orlando, FL (US); Pritesh Patel, Orlando, FL (US); Balavignesh Thirumalainambi, Ridgewood, NJ (US); Srini Surendranath, Westerville, OH (US); Raj Toleti, Orlando, FL (US)

(73) Assignee: AndorHealth, LLC, Orlando, FL (US)

(21) Appl. No.: 18/374,228

(22) Filed: Sep. 28, 2023

Related U.S. Application Data
(60) Provisional application No. 63/410,688, filed on Sep. 28, 2022.

(51) Int. Cl. G16H 40/20; G06Q 10/047

(52) U.S. Cl. CPC G16H 40/20; G06Q 10/047; G16H 50/30

(57) ABSTRACT

A system can include a location data module configured to receive location data, the location data including patient location information associated with a plurality patients and care team member location information associated with one or more care team members, and a route module operatively connected to the location data module and configured to determine a visitation route for each of the one or more care team members to visit the plurality of patients based on the location data.

### CROSS-REFERENCE TO RELATED APPLICATIONS

This application claims priority to and the benefit of U.S. Provisional Application No. 63/410,688, filed Sep. 28, 2022, the entire contents of which are herein incorporated by reference in their entirety.

### FIELD

This disclosure relates to care provider routing systems.

### BACKGROUND

In the current workflow, software solutions to facilitate Hospital at Home programs are limited in their functionality, which restricts the benefits of home care for patients. Today due to lack of space, hospitals are incentivized to send patients home to continue treatment. Reducing the number of patients kept in the hospital at any given time will prevent transmission of hospital infections and create more space for high acuity patients. In addition to this, in the case of an epidemic and to prepare sufficiently for a similar disaster, hospitals should implement policies by which they can treat as many patients as possible while minimizing the risk of transmission between patients and to healthcare practitioners. By establishing infrastructure to care for patients at home, hospitals will increase their maximum patient capacity and reduce the inherent risk of transmission of infectious diseases.

Such conventional methods and systems have generally been considered satisfactory for their intended purpose. However, there is still a need in the art for improvements. The present disclosure provides a solution for this need.

### SUMMARY

In accordance with at least one aspect of this disclosure, a system can include a location data module configured to receive location data, the location data including patient location information associated with a plurality patients and care team member location information associated with one or more care team members, and a route module operatively connected to the location data module and configured to determine a visitation route for each of the one or more care team members to visit the plurality of patients based on the location data. In certain embodiments, the route module is configured to output the visitation route to a respective care team member device.

In certain embodiments, the care team member location information can be or include care team member geolocation data. In certain embodiments, the patient location information can be or include patient geolocation data.

In certain embodiments, the care team member location information can be associated with a plurality of care team members. In certain embodiments, the route module can be configured to determine a distribution of patients between the plurality of care team members to determine the visitation route for each of the plurality of care team members such that the distribution of patients reduces or minimizes a total time and/or total distance of aggregate visitation routes.

In certain embodiments, the system can further include a relevant information module connected to the route module and configured to provide relevant information to the route module. The route module can be configured to determine a patient priority score for each patient based on the relevant information and/or the location data, create an ordered patient list based on the priority score, and determine the visitation route for each of the one or more care team members based on the ordered patient list. In certain embodiments, the relevant information can include one or more of patient symptom information, traffic data, medical information completeness, one or more risk factors, and/or patient medical record data.

In certain embodiments, the route module can be configured to dynamically update the priority score, the ordered patient list, and the visitation route of each of the one or more care team members dynamically based on updated relevant information and/or when a change is made to the relevant information. In certain embodiments, the priority score of each patient can be based upon a severity of a medical condition and geographic location.

In accordance with at least one aspect of this disclosure, a computer implemented method can include receiving a patient list having a plurality of patients to be visited, receiving one or more symptoms associated with each patient on the patient list, and receiving patient location information associated with each patient, and determining a severity score for each patient on the patient list based on the one or more symptoms of each patient. The method can further include determining a travel score for each patient based on the patient location information, calculating a patient priority score for each patient on the patient list by combining the severity score with the travel score, and creating an ordered patient list having the plurality of patients ordered based on the priority score of each patient. The method can further include generating a visitation route for a care team member to visit two or more of the plurality of patients using the ordered patient list to determine an order of visitation of the two or more of the plurality of patients, and outputting the visitation route to a device associated with the care team member.

In certain embodiments, receiving the patient location information includes receiving patient geolocation. The method further includes determining a time-to-arrival at each patient's geolocation, wherein determining the travel score includes using the time-to-arrival. In certain embodiments, determining the patient priority score can include providing a lower priority for farther patients. In certain embodiments, determining the patient priority score includes providing a higher priority to patients with more severe symptoms. In certain embodiments, generating the visitation route includes generating the most time efficient route for the care team member based on care team member location information. In certain embodiments, the method can include updating the visitation route based on new information and/or after every visit to reroute the care team member if there is a change in a patient's priority.

In accordance with at least one aspect of this disclosure, a non-transitory computer readable medium can include computer executable instructions configured to cause a computer to perform a method. The method can include any suitable method disclosed herein, e.g., described above.

These and other features of the embodiments of the subject disclosure will become more readily apparent to those skilled in the art from the following detailed description taken in conjunction with the drawings.

### BRIEF DESCRIPTION OF THE DRAWINGS

So that those skilled in the art to which the subject disclosure appertains will readily understand how to make and use the devices and methods of the subject disclosure without undue experimentation, embodiments thereof will be described in detail herein below with reference to certain figures, wherein:

FIG. 1 is a schematic diagram of an embodiment of a system in accordance with this disclosure.

FIG. 2 illustrates an embodiment of a graphical user interface of an embodiment of a system in accordance with this disclosure, showing a provider view wherein a provider can assign members to teams and facilities while also providing a description of each.

FIG. 3 illustrates an embodiment of a graphical user interface of an embodiment of a system in accordance with this disclosure, showing a provider view for specifying triggers, outreach type, alert time, and configuring which team members and/or teams receive notifications.

FIGS. 4A, 4B, and 4C illustrate an embodiment of a graphical user interface of an embodiment of a system in accordance with this disclosure, showing care team alerts, e.g., showing a screen in which care team member receives notifications about new patients (e.g., via text message as shown in FIG. 4A), accepts or declines the new patient (e.g., as shown in FIG. 4B), and views care team information (e.g., as shown in FIG. 4C).

FIGS. 5A and 5B illustrate an embodiment of a priority score calculation algorithm for a first patient (FIG. 5A) and a second patient (FIG. 5B).

FIG. 6 illustrates an embodiment of a graphical user interface of an embodiment of a system in accordance with this disclosure, showing a prioritized list of patients and if their in-person and virtual visits have been completed.

FIG. 7 illustrates an embodiment of a graphical user interface of an embodiment of a system in accordance with this disclosure, showing a patient dashboard welcome screen for a patient.

FIGS. 8A and 8B illustrate an embodiment of a graphical user interface of an embodiment of a system in accordance with this disclosure, showing a dynamic patient itinerary (e.g., a visitation route on a mobile device of a care team member).

FIG. 9 illustrates an embodiment of a graphical user interface of an embodiment of a system in accordance with this disclosure, showing an embodiment of a traffic update notification (e.g., a text message push notification) to alert a care team member of traffic delays in the visitation route.

FIG. 10 illustrates an embodiment of a graphical user interface of an embodiment of a system in accordance with this disclosure, showing a survey to be completed by patients after completing their hospital-at-home regiment.

FIGS. 11A, 11B, and 11C illustrate an embodiment of a graphical user interface of an embodiment of a system in accordance with this disclosure, showing a patient discharge notification (e.g., a text message push notification as shown in FIG. 11A sent to a care team member's device), a patient's information (e.g., as shown FIG. 11B accessed by clicking a link in the discharge notification), and a summary of the patient's discharge for both the patient and provider (e.g., as shown in FIG. 11C).

### DETAILED DESCRIPTION

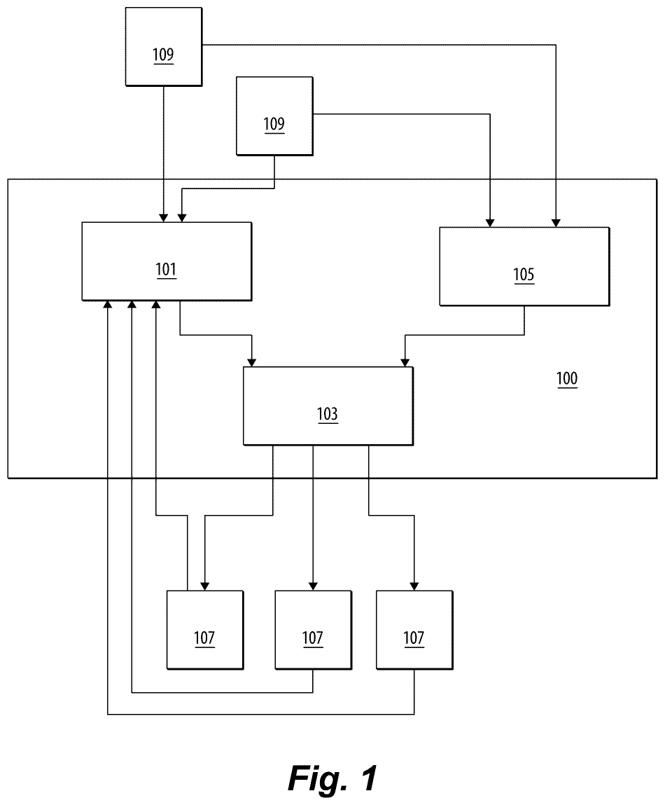

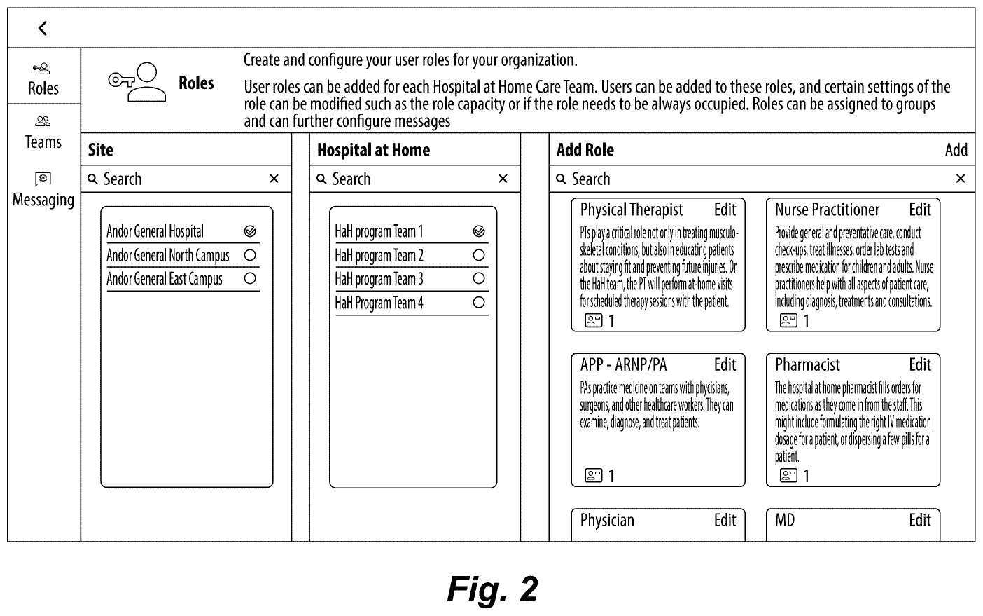

Reference will now be made to the drawings wherein like reference numerals identify similar structural features or aspects of the subject disclosure. For purposes of explanation and illustration, and not limitation, an illustrative view of an embodiment of a system in accordance with the disclosure is shown in FIG. 1 and is designated generally by reference character 100. Other views, embodiments, and/or aspects of this disclosure are illustrated in FIGS. 2-11C.

In accordance with at least one aspect of this disclosure, referring to FIG. 1, a system 100 can include a location data module 101 configured to receive location data. The location data can include patient location information associated with a plurality patients (e.g., hospital at home patients) and care team member location information associated with one or more care team members (e.g., doctors, nurses, etc.). The system 100 can include a route module 103 operatively connected to the location data module 101 and configured to determine a visitation route (e.g., information configured to provide driving directions for a vehicle in a mobile device map application) for each of the one or more care team members to visit the plurality of patients (e.g., collectively between all care team members where there are a plurality of care team members) based on the location data.

In certain embodiments, the route module 103 can be configured to output the visitation route to a respective care team member device 107 (e.g., a mobile smart phone or tablet, an in-vehicle navigation system, etc.). The visitation route for each care team member can be or include a plurality of destinations (e.g., GPS coordinates, addresses, or any other suitable information for navigation) listed and ordered by patient priority based on a priority score, for example. The list of destinations can be input into navigational software as successive waypoints, for example. The visitation route can be or include any suitable data configured to cause route creation on any suitable device.

In certain embodiments, the care team member location information can be or include care team member geolocation data (e.g., device 107 location information such as GPS location, or based on an assumed or selected starting location). In certain embodiments, the patient location information can be or include patient geolocation data (e.g., based on a known address or GPS information of a device 109 associated with a respective patient).

In certain embodiments, the care team member location information can be associated with a plurality of care team members (e.g., in that it can include location information for multiple care team member devices 107 (e.g., as shown in FIG. 1). For example, in certain embodiments, the route module 103 can be configured to determine a distribution of patients between the plurality of care team members to determine the visitation route for each of the plurality of care team members, e.g., such that the distribution of patients reduces or minimizes a total time and/or total distance of aggregate visitation routes. In this regard, the route module 103 can see where each care team member is, and determine a visitation route for each such that each patient is included on at least one (e.g., only one) of the care team members visitation routes. The total time and/or distance of the visitation routes of all care team members can be added and minimized using the location information and/or other relevant information (e.g., described below). For example, care team members that are located closer to certain patients can be assigned those patients to visit to minimize travel time and/or distance.

In certain embodiments, the system 100 can further include a relevant information module 105 connected to the route module 103 and configured to provide relevant information to the route module 103. The route module 103 can be configured to determine a patient priority score for each patient based on the relevant information and/or the location data, create an ordered patient list based on the priority score, and determine the visitation route for each of the one or more care team members based on the ordered patient list.

In certain embodiments, the relevant information can include one or more of patient symptom information, traffic data, medical information completeness, one or more risk factors (e.g., age, illness type), and/or patient medical record data. Any other suitable relevant information affecting travel time or patient safety/urgency is contemplated herein.

In certain embodiments, the route module 103 can be configured to dynamically update the priority score, the ordered patient list, and the visitation route of each of the one or more care team members dynamically based on updated relevant information (e.g., updated traffic information in real time) and/or when a change is made to the relevant information (e.g., a patient inputs more symptom information). In certain embodiments, for example, the priority score of each patient can be based upon a severity of a medical condition and geographic location.

It is contemplated that the system 100 can be access via the internet and/or in any other suitable manner. Any suitable type of wired, wireless, direct, or indirect connection to the system 100 is contemplated herein. The system 100 can include any suitable graphical user interfaces (GUIs) accessible to the patients (e.g., a patient portal for inputting symptom information and a care team member portal for receiving patient information and visitation routes). The system 100 can be embodied as a software as a service (SAAS) application and/or can be entirely or partially implemented as a locally downloadable software (e.g., a local navigation application connected to the SAAS application for receiving visitation routes).

Certain embodiments can include artificial intelligence and/or machine learning for intelligently providing routing based on the provided location and/or relevance information. In certain embodiments, certain care team members may be limited to certain patients either manually or based on qualifications of the care team member (e.g., a cardiologist may be required to visit a patient with congestive heart failure).

In accordance with at least one aspect of this disclosure, a computer implemented method can include receiving a patient list having a plurality of patients to be visited, receiving one or more symptoms associated with each patient on the patient list, and receiving patient location information associated with each patient, and determining a severity score for each patient on the patient list based on the one or more symptoms of each patient. The method can further include determining a travel score for each patient based on the patient location information, calculating a patient priority score for each patient on the patient list by combining the severity score with the travel score, and creating an ordered patient list having the plurality of patients ordered based on the priority score of each patient. The method can further include generating a visitation route for a care team member to visit two or more of the plurality of patients using the ordered patient list to determine an order of visitation of the two or more of the plurality of patients, and outputting the visitation route to a device associated with the care team member.

In certain embodiments, receiving the patient location information includes receiving patient geolocation. The method further includes determining a time-to-arrival at each patient's geolocation, wherein determining the travel score includes using the time-to-arrival. In certain embodiments, determining the patient priority score can include providing a lower priority for farther patients. In certain embodiments, determining the patient priority score includes providing a higher priority to patients with more severe symptoms. In certain embodiments, generating the visitation route includes generating the most time efficient route for the care team member based on care team member location information. In certain embodiments, the method can include updating the visitation route based on new information and/or after every visit to reroute the care team member if there is a change in a patient's priority.

In accordance with at least one aspect of this disclosure, a non-transitory computer readable medium can include computer executable instructions configured to cause a computer to perform a method. The method can include any suitable method and/or portions thereof disclosed herein, e.g., described above.

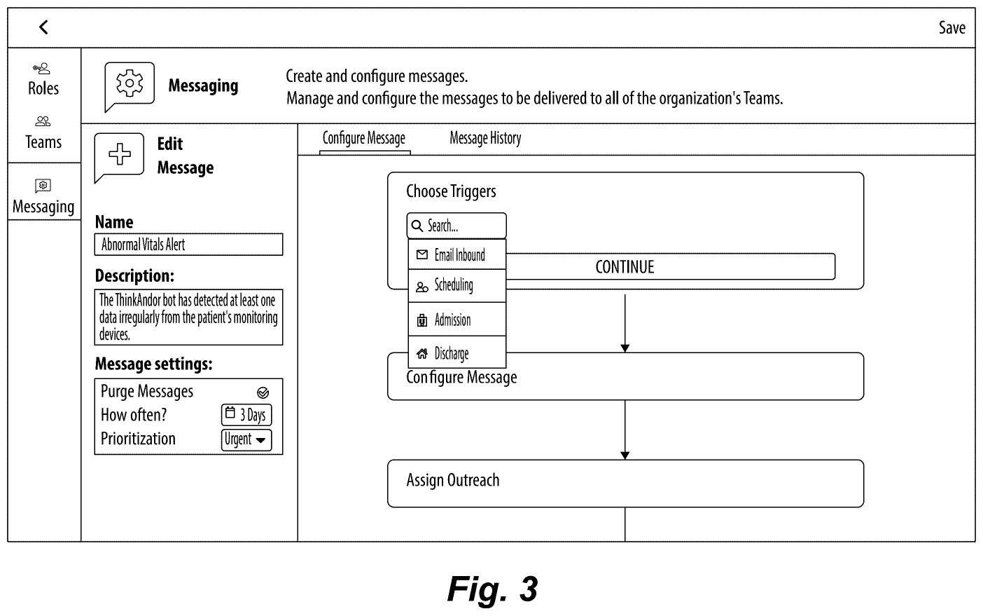

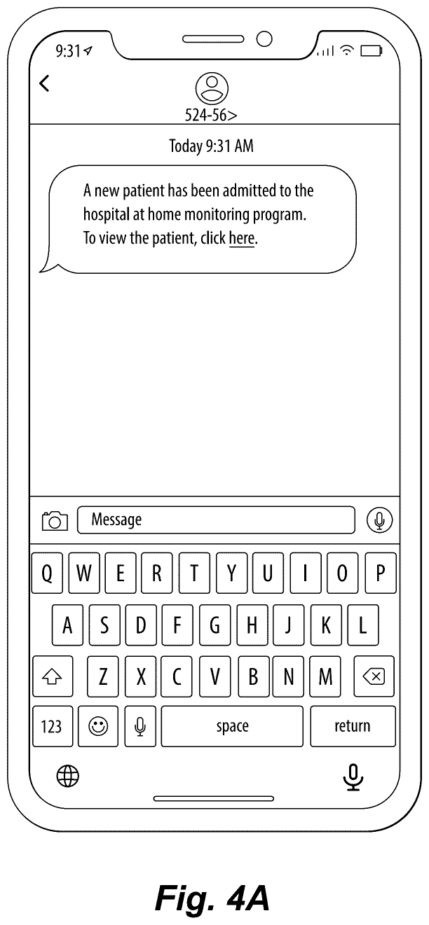

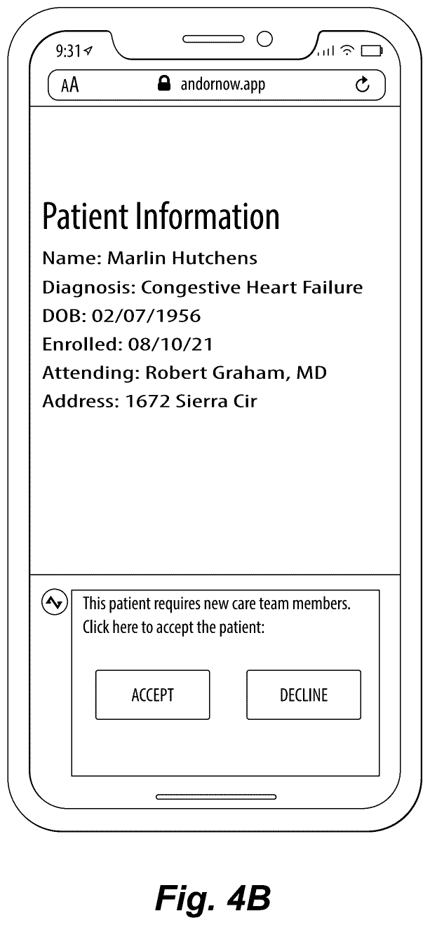

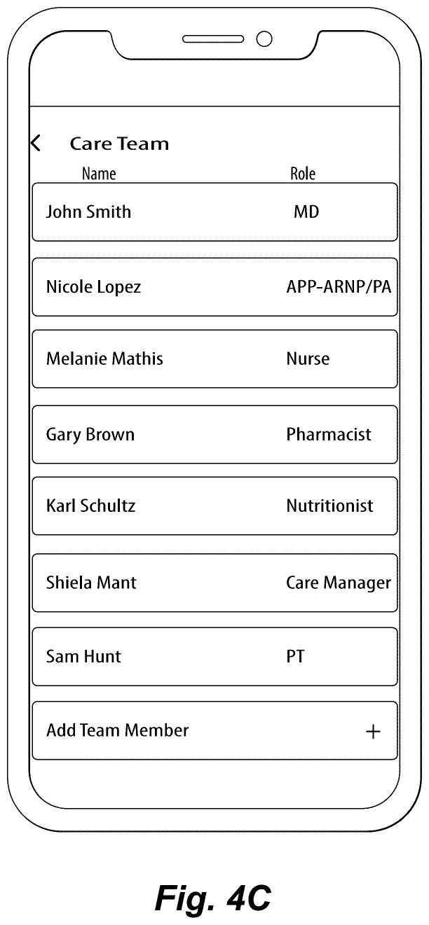

FIG. 2 illustrates team/role creation, for example, showing a provider view of assigning members to teams and facilities while also providing a description of each. FIG. 3 illustrates team message creation, showing provider view for specifying triggers, outreach type, alert time, and configuring which team members and/or teams receive notifications. FIGS. 4A, 4B, and 4C illustrate care team alerts, showing screens in which care team can receive notifications about new patients, accept/decline them, and view care team information.

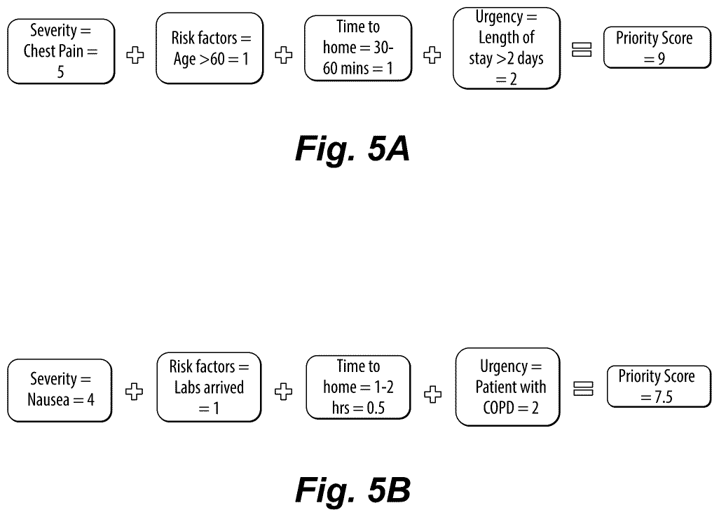

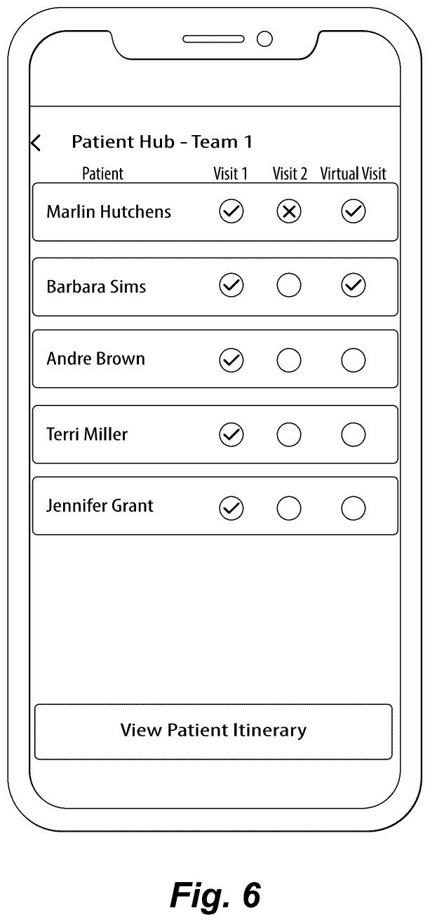

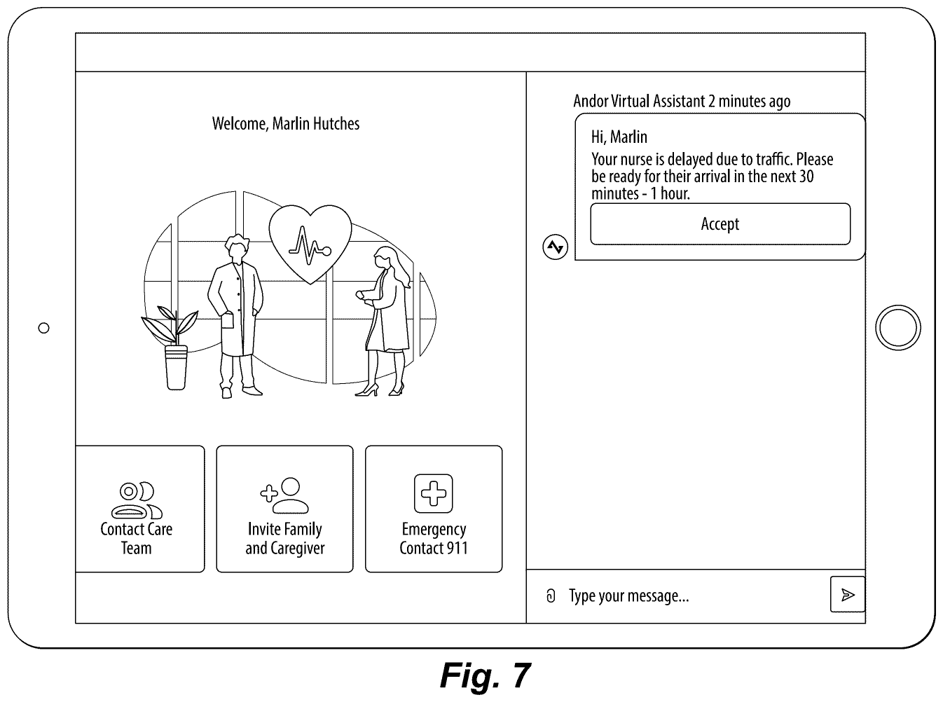

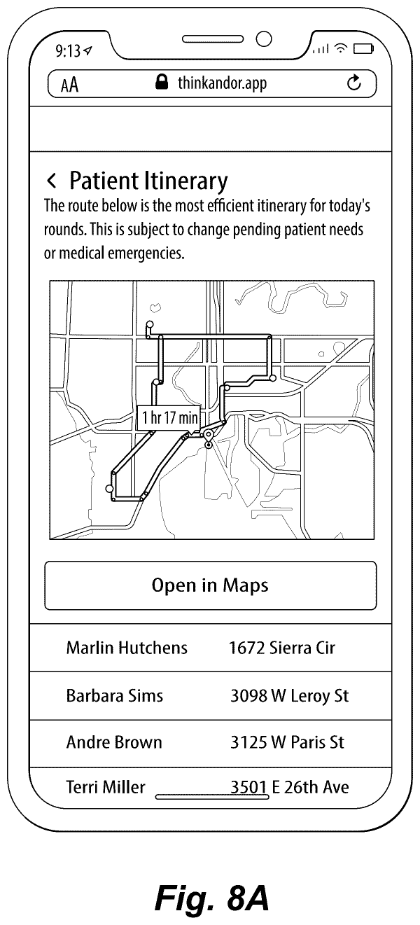

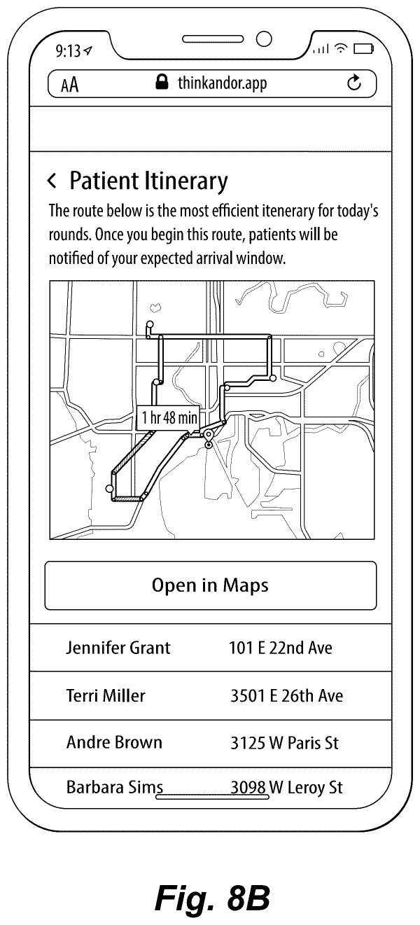

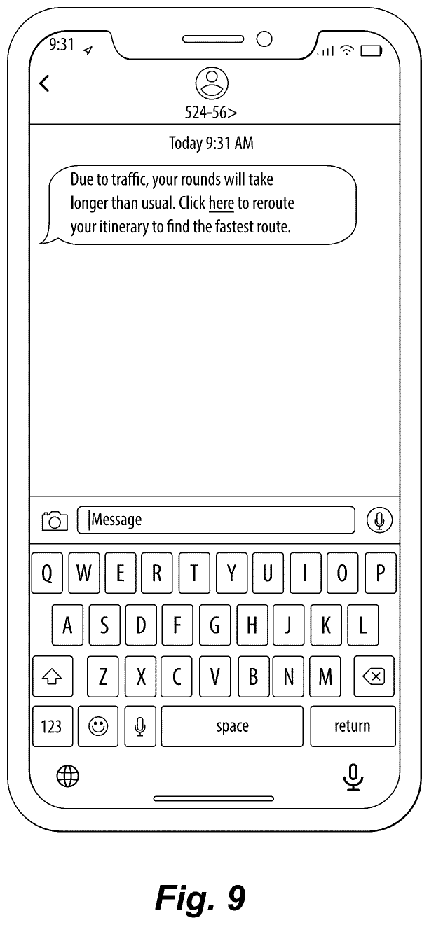

FIGS. 5A and 5B illustrate an algorithm flowchart, showing a visual representation of a filtration system that the system uses to prioritize patients. FIG. 6 illustrates a patient itinerary, showing a prioritized list of patients and if their in-person and virtual visits have been completed. FIG. 7 illustrates a patient dashboard, displaying a welcome screen for a patient. FIGS. 8A and 8B illustrate a dynamic patient itinerary, showing an embodiments wherein the system sorts patient rounding based on location and accounts for traffic conditions in real-time. FIG. 9 illustrates traffic update notifications, showing a push notifications to alert the care team member of traffic delays in rounding schedules.

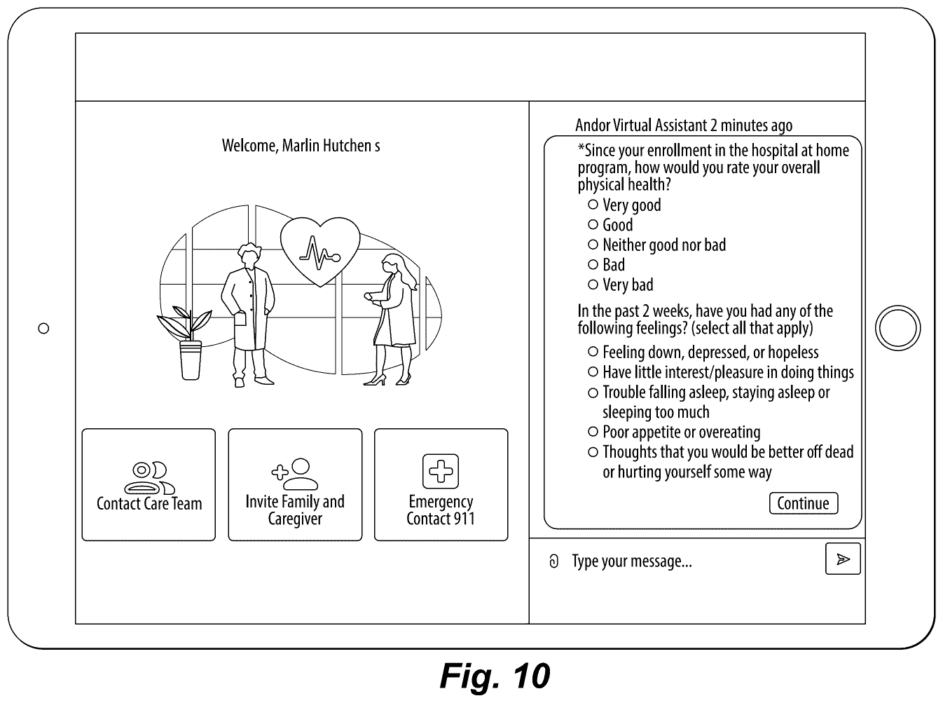

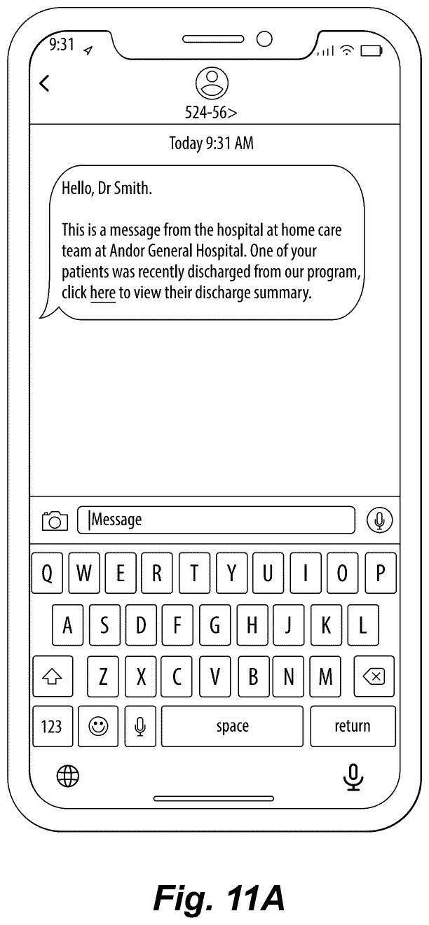

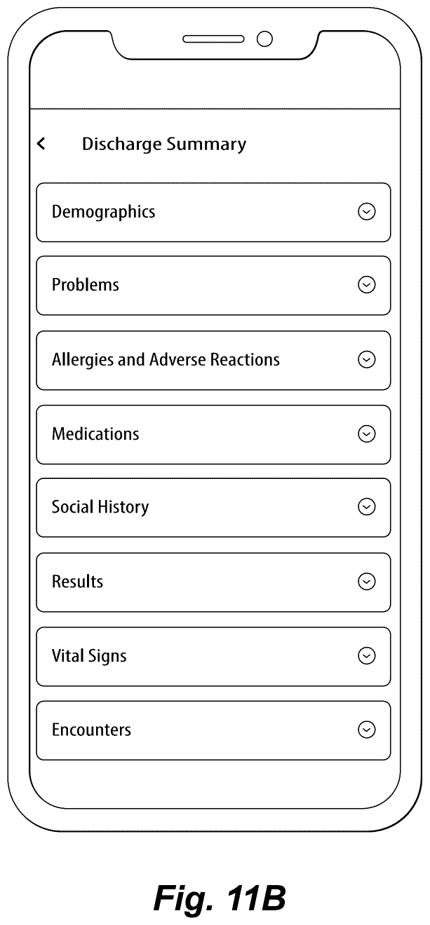

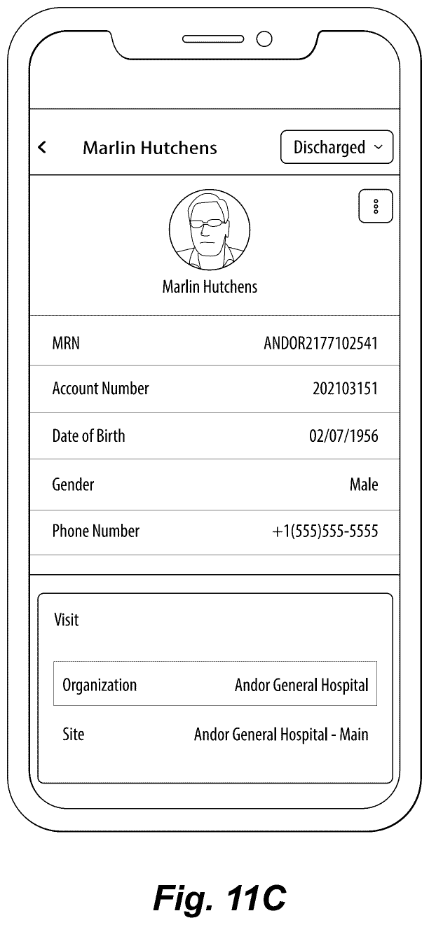

FIG. 10 illustrates a survey to be completed by patients after completing their hospital-at-home regiment. FIGS. 11A, 11B, and 11C illustrate a patient discharge notification sent to a care team member device, a patient's information, and a summary of the patient's discharge for both the patient and provider, respectively.

Certain embodiments can provide an intelligent Hospital at Home care team routing solution. Embodiments disclosed herein can be applied in the healthcare field, e.g., more specifically to Hospital at Home technology for workflow optimization.

Embodiments of a process and system can streamline the procedures involved with attending to patients while they recover at home. Members of the care team can be given a route to see their patients that is formulated based on the distance between each patient and their specific conditions. In instances where patients incur additional symptoms or problematic test results, their priority can be elevated, and they can be seen sooner. Allowing practitioners to more effectively treat patients who are not staying in the hospital can reduce margins and increase efficiency such that they can see more patients in the same amount of time while providing the best possible care for the patients. Currently, when a patient's care team wants to keep them for observation, they can stay in the hospital accruing bills for constant care and prohibiting other potential patients from receiving care at the facility. This reduces the number of patients a facility can actively service. Moreover, by implementing a streamlined process where members of the care team can round their patients outside of the hospital, patients can be more likely to follow care instructions and medication adherence to enable speedy recovery.

Embodiments can include one or more hardware and/or software module(s) configured to perform and function, method, algorithm, etc. disclosed herein. Certain embodiments allow administrators to create care teams and configure roles for Hospital at Home care. Providers can assign members to teams and facilities while also providing a description of each (e.g., as shown in FIG. 2). Through the system, providers can create messages and configure notifications. They can specify triggers, outreach type, alert time, and configure which team members and/or teams receive notifications (e.g., as shown in FIG. 3). Consequently, care team members can be alerted of updates in the patient list (e.g., additional patients, workflow changes, etc.; e.g., as shown in FIGS. 4A-4C). Alerts regarding patient statuses, lab results, and traffic conditions are aggregated in a real-time algorithm that establishes a fluctuating priority score for each patient that combines the acuity of their symptoms and their distance from the traveling practitioners. Embodiments of a system can also include a module configured to take account of the interactions between certain diagnoses and symptoms. For example, assume symptom X is a serious symptom within the context of congestive heart failure, however less so in the context of pneumonia. For this reason, a patient with CHF (Congestive Heart Failure) reporting symptom X can garner a higher priority score than a patient with pneumonia also reporting symptom X.

Embodiments can include a module that utilizes an algorithm that creates a dynamic patient list based on priority for care team members to visit patients on an efficient route. The priority of each patient can be based upon the severity of their condition and their geographic location. The factors in consideration can be a myriad of symptoms, e.g., the traffic in the area, and whether labs, prescriptions, etc., have arrived to the patient. In this regard, the module(s) can be operatively connected to any suitable geolocation data source, traffic source, weather source, etc., as well as the a medical information system, to receive such data and output a visitation route (e.g., the most time or distance efficient route) or each care team member.

An embodiment of an algorithm that can be implemented by one or more module(s) is described below. The algorithm can begin with an initial list of Hospital at Home patients. Next, the algorithm can filter them through a list of symptoms they might be experiencing to assign them an initial severity score. Determined by medical professionals, each symptom in the algorithm can be assigned a numerical value based on its acuity. The algorithm can also take into consideration how a combination of specific symptoms may increase severity. Following these steps, the total patient priority score can then calculated by combining the severity of the patient symptoms and a numerical value based on the time it takes to arrive at the patient's house. To minimize transit times, farther patients can be given lower priority. Additionally, the algorithm can consider if the necessary elements are ready for the appointment to take place under the risk factors. This includes the age of the patient and whether the patient's labs are ready and if certain prescriptions/medications have been distributed to the patient. Similarly, these conditions can be be given a numerical value to be added to the patient' s total priority score since a lack of preparedness causes inefficiency (e.g., as shown in FIGS. 5A and 5B). After taking all the factors into consideration, the algorithm can tally a total priority score.

The patients can be displayed in order of greatest to least severity for the care team (e.g., as shown in FIG. 6), and the most efficient and timely route can be displayed, scheduling their day. The algorithm can be updated whenever new information comes through and after every visit to reroute the care team if there is a change in a patient's priority (e.g., as shown in FIG. 7). Based upon the traffic patterns for a given day, the care team can be notified of changes in traffic conditions which impact their itinerary (e.g., as shown in FIGS. 8A and 8B). The system can provide a manual button to open the route in a local maps application, e.g., as shown.

Additionally, the care team can also be notified if there is time to have a virtual appointment with patients on the way to their in-person visits for the day. The algorithm can take into account real-time updates, such as the ever-changing traffic routes, new arrival of lab and test results, and changes in symptoms. This can send updates to the care team as the final list of prioritized patients may change. The algorithm can be implemented by any suitable number and/or distribution of hardware and/or software module(s) (e.g., on a single server as a SAAS).

Each patient can be provided with a tablet where they can contact their care team, invite family members/caregivers, and contact 9-1-1 (e.g., as shown in FIGS. 7 and 10). There can also be a chat feature where the patient can receive notifications that can include updates about their care team's arrival time, complete customer satisfaction surveys, contact their care team, etc. (e.g., as shown in FIG. 7).

In certain embodiments, Hospital at Home can include a five-day care plan. The patient can exit the in-person emergency department (ED) setting on the first day, and the providers can receive notifications about a newly admitted Hospital at Home patient. Here, they can accept/decline the patient and view the other members of the care team (e.g., as shown in FIGS. 4A and 4B). On day two, three, and four, remote patient monitoring (RPM) data can be gathered (e.g., for blood pressure (BP), pulse oximeter data, and weight), the patient can complete daily questionnaires, and care team members can complete two in-person visits and a virtual visit. In addition to the previous descriptions, on the fifth day, the patient can be sent a discharge notification with information and can then transition to an RPM program. Any other suitable timeline and/or program structure is contemplated herein.

Care team providers can receive alerts with lab reports, vitals, and other pertinent patient information. Within the solution, a chat feature enables providers to discuss patients and send/receive patient information amongst team members while complying with HIPPA guidelines. Additionally, this facilitates the home care process, optimizing Hospital at Home. Finally, when patients graduate from their home health care, the one or more module(s) (e.g., comprising a software system) can enable providers to send discharge summaries (e.g., as shown in FIGS. 11B, and 11C), so patient recovery is assured along with an efficient home visit.

Hospital at home programs that enable patients to receive hospital-level care in the comfort of their homes have flourished in countries with single-payer health systems. Such systems are beginning to be used in many countries. Moreover, it has been established that home hospitalizations can be safer, cheaper, and more effective than institutional care. This becomes especially important for patients who are vulnerable to hospital-acquired infections and other complications of inpatient care. In order to meet this demand for hospital at home care, the embodiments disclosed herein can include an intelligent, healthcare software that can allow care team members from a hospital to provide effective Hospital at Home care, dynamically update patient itineraries based on patient location, priority, eligibility, etc. The system can enable healthcare providers to assign care team members with pre-defined roles and dispatches notifications to update care team members on patient conditions. The solution can not only allow the care team members from a hospital to provide effective hospital at home care, but also can intelligently determine the priority of the patient to be seen first and can utilize virtual visits technology when travel to the patient is not necessary.

In certain embodiments, a system can include a data module configured to receive geolocation data from each patient and each care team member, and a route module configured to determine a most time efficient visitation route for each care team member and patient. For example, based on the location of each care team member, the route module can decide which care team member is to visit which patients so that the distribution of patients provide the most time or distance efficient visitation schedule. The data module can receive any other suitable relevant information (e.g., symptoms, traffic, medical data, etc.) and determine a priority of the list for a care team member.

In certain embodiments, a computer implemented method for organizing patient lists for hospital-at-home care team members can include creating a patient itinerary for each care team member to perform patient rounding and visits, wherein creating the patient itinerary is based on one or more of geolocation of each patient and each team member, traffic conditions, visit eligibility, and/or patient severity. In certain embodiments, the method can include receiving geolocation information about each patient, wherein creating the patient itinerary includes sorting patients on shortest geographical distance. In certain embodiments, the method can include receiving the traffic conditions, wherein creating the patient itinerary includes optimizing care team travel. In certain embodiments, the method can include using a database which weighs the severity of patient conditions to contribute to an optimized patient itinerary. In certain embodiments, creating the patient itinerary can include determining the status of patients' labs and medication and determining whether a patients' visit should instead be a virtual visit.

Certain embodiments can include a method in which Hospital at Home care teams can optimize care team collaboration and ADT notifications in a Hospital at Home environment. In certain embodiments, a system can include one or more modules which enables administrators to create and configure roles utilizing taxonomy, share patient information, and discuss patient condition, wherein they are enabled to send/receive notifications between teams and team members, while complying with HIPPA guidelines. Based on the configuration and taxonomy the system can dynamically assign roles to individuals who are actively utilizing the system at a given particular time and location. In certain embodiments, the system can comprise an ADT notification system which notifies at-home patients of updates for traveling care team members, wherein patients can be notified of traffic conditions, travel time updates, and virtual visit appointments. In certain embodiments, a system can include one or more modules configured to perform any of the methods disclosed herein.

Embodiments can include any suitable computer hardware and/or software module(s) to perform any suitable function (e.g., as disclosed herein). Any suitable method(s) or portion(s) thereof disclosed herein can be performed on and/or by any suitable hardware and/or software module(s).

As will be appreciated by those skilled in the art, aspects of the present disclosure may be embodied as a system, method or computer program product. Accordingly, aspects of this disclosure may take the form of an entirely hardware embodiment, an entirely software embodiment (including firmware, resident software, micro-code, etc.), or an embodiment combining software and hardware aspects, all possibilities of which can be referred to herein as a “circuit,” “module,” or “system.” A “circuit,” “module,” or “system” can include one or more portions of one or more separate physical hardware and/or software components that can together perform the disclosed function of the “circuit,” “module,” or “system”, or a “circuit,” “module,” or “system” can be a single self-contained unit (e.g., of hardware and/or software). Furthermore, aspects of this disclosure may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of this disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of this disclosure may be described above with reference to flowchart illustrations and/or block diagrams of methods, apparatus (systems) and computer program products according to embodiments of this disclosure. It will be understood that each block of any flowchart illustrations and/or block diagrams, and combinations of blocks in any flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in any flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified herein.

Those having ordinary skill in the art understand that any numerical values disclosed herein can be exact values or can be values within a range. Further, any terms of approximation (e.g., “about”, “approximately”, “around”) used in this disclosure can mean the stated value within a range. For example, in certain embodiments, the range can be within (plus or minus) 20%, or within 10%, or within 5%, or within 2%, or within any other suitable percentage or number as appreciated by those having ordinary skill in the art (e.g., for known tolerance limits or error ranges).

The articles “a”, “an”, and “the” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

Any suitable combination(s) of any disclosed embodiments and/or any suitable portion(s) thereof are contemplated herein as appreciated by those having ordinary skill in the art in view of this disclosure.

The embodiments of the present disclosure, as described above and shown in the drawings, provide for improvement in the art to which they pertain. While the subject disclosure includes reference to certain embodiments, those skilled in the art will readily appreciate that changes and/or modifications may be made thereto without departing from the spirit and scope of the subject disclosure.

What is claimed is:

1. A system, comprising:
a location data module configured to receive location data, the location data including patient location information associated with a plurality patients and care team member location information associated with one or more care team members; and
a route module operatively connected to the location data module and configured to determine a visitation route for each of the one or more care team members to visit the plurality of patients based on the location data.

2. The system of claim 1, wherein the route module is configured to output the visitation route to a respective care team member device.

3. The system of claim 1, wherein the care team member location information is or includes care team member geolocation data.

4. The system of claim 3, wherein the patient location information is or includes patient geolocation data.

5. The system of claim 1, wherein the care team member location information is associated with a plurality of care team members, wherein the route module is configured to determine a distribution of patients between the plurality of care team members to determine the visitation route for each of the plurality of care team members such that the distribution of patients reduces or minimizes a total time and/or total distance of aggregate visitation routes.

6. The system of claim 1, further comprising a relevant information module connected to the route module and configured to provide relevant information to the route module.

7. The system of claim 6, wherein the route module is configured to:
determine a patient priority score for each patient based on the relevant information and/or the location data;
create an ordered patient list based on the priority score; and
determine the visitation route for each of the one or more care team members based on the ordered patient list.

8. The system of claim 7, wherein the relevant information includes one or more of patient symptom information, traffic data, medical information completeness, one or more risk factors, and/or patient medical record data.

9. The system of claim 8, wherein the route module is configured to dynamically update the priority score, the ordered patient list, and the visitation route of each of the one or more care team members dynamically based on updated relevant information and/or when a change is made to the relevant information.

10. The system of claim 9, wherein the priority score of each patient is based upon a severity of a medical condition and geographic location.

11. A computer implemented method, comprising:
receiving a patient list having a plurality of patients to be visited;
receiving one or more symptoms associated with each patient on the patient list;
receiving patient location information associated with each patient;
determining a severity score for each patient on the patient list based on the one or more symptoms of each patient;
determining a travel score for each patient based on the patient location information;
calculating a patient priority score for each patient on the patient list by combining the severity score with the travel score;
creating an ordered patient list having the plurality of patients ordered based on the priority score of each patient;
generating a visitation route for a care team member to visit two or more of the plurality of patients using the ordered patient list to determine an order of visitation of the two or more of the plurality of patients; and
outputting the visitation route to a device associated with the care team member.

12. The computer implemented method of claim 11, wherein receiving the patient location information includes receiving patient geolocation, and wherein the method further includes determining a time-to-arrival at each patient's geolocation, wherein determining the travel score includes using the time-to-arrival.

13. The computer implemented method of claim 12, wherein determining the patient priority score includes providing a lower priority for farther patients.

14. The computer implemented method of claim 13, wherein determining the patient priority score includes providing a higher priority to patients with more severe symptoms.

15. The computer implemented method of claim 14, wherein generating the visitation route includes generating the most time efficient route for the care team member based on care team member location information.

16. The computer implemented method of claim 15, further comprising updating the visitation route based on new information and/or after every visit to reroute the care team member if there is a change in a patient's priority.

17. A non-transitory computer readable medium, comprising computer executable instructions configured to cause a computer to perform a method, the method, comprising:
receiving a patient list having a plurality of patients to be visited;
receiving one or more symptoms associated with each patient on the patient list;
receiving patient location information associated with each patient;
determining a severity score for each patient on the patient list based on the one or more symptoms of each patient;
determining a travel score for each patient based on the patient location information;
calculating a patient priority score for each patient on the patient list by combining the severity score with the travel score;
creating an ordered patient list having the plurality of patients ordered based on the priority score of each patient;
generating a visitation route for a care team member to visit two or more of the plurality of patients using the ordered patient list to determine an order of visitation of the two or more of the plurality of patients; and
outputting the visitation route to a device associated with the care team member.

18. The non-transitory computer readable medium of claim 17, wherein receiving the patient location information includes receiving patient geolocation, and wherein the method further includes determining a time-to-arrival at each patient's geolocation, wherein determining the travel score includes using the time-to-arrival.

19. The non-transitory computer readable medium of claim 18, wherein determining the patient priority score includes providing a lower priority for farther patients.

20. The non-transitory computer readable medium of claim 19, wherein determining the patient priority score includes providing a higher priority to patients with more severe symptoms.
